(19) United States
(12) Patent Application Publication
(10) Pub. No.: US 2015/0320445 A1
(43) Pub. Date: Nov. 12, 2015

(54) Technique and Apparatus for extracting ingrown hairs that cause razor bumps

(71) Applicants: Derrick Todd Wynn, Marietta, GA (US); Samuel James, Suffolk, VA (US)

(72) Inventors: Derrick Todd Wynn, Marietta, GA (US); Samuel James, Suffolk, VA (US)

(21) Appl. No.: 14/275,751

(22) Filed: May 12, 2014

(51) Int. Cl. A61B 17/50

(52) U.S. Cl. CPC A61B 17/50; A61B 2017/00752

(57) ABSTRACT

This is an apparatus for pulling out ingrown hairs that cause razor bumps, with an application of hot water and soap lather or shaving foam. This opens the pores and loosens the ingrown hairs for extracting. The textured convex head with rounded corners, allows for a fluent motion on various contoured surfaces of the human body that is susceptible to ingrown hairs. The width of the textured head has enough surface area to capture the multiple ingrown hairs and extracting them in a minimal amount of time.

### BACKGROUND OF THE INVENTION

Pseudo-folliculitis barbae, also called an ingrown hair or razor bump, is inflicted upon any race or gender and it can happen on any part of the body, when a razor is used on sensitive skin with curly hair. After the hair has been shaved, it begins to grow back and curly hair tends to curl into the skin instead of straight out of the follicle, leading to an inflammation reaction. PFB can make the skin look itchy and red, and in some cases, it can even look like pimples. This is especially problematic for African American men and women who have naturally coarse or tightly curling hair.

In the past, home treatment usually involved removing of the ingrown hair with tweezers or needles. Countless hours are spent trying to pull out Individual hairs properly, when none of these worked, it caused infections. It was necessary to find something that was less painful and takes less time. When home remedies do not work, individuals were turning to topical applications with chemicals that caused rashes and irritated the skin.

Razor bumps have always been a major problem and people will use different techniques to try conquer them, such as plucking, waxing, and electrolysis, but they all cause ingrown hairs.

To overcome frustration with razor bumps, there remains a need for an improved technique and apparatus capable of extracting multiple ingrown hairs simultaneously in less time and with less pain. To accomplish this, hot water and soap lather or shaving foam is applied to the affected area and then the skin is exfoliated. This can be done in between shaving, to keep the ingrown hairs from growing back into the skin.

### BRIEF SUMMARY OF THE INVENTION

An apparatus designed to pull out multiple ingrown hairs from the skin simultaneously, by exfoliation. It is used in conjunction with applying hot water on the skin, to soften the ingrown hairs and soap lather or shaving foam to make it feasible for extracting. The apparatus is then applied, by moving in an up and downward or side to side motion, against the grain of the hair growth, to pull out the ingrown hairs.

### BRIEF DESCRIPTION OF SEVERAL VIEWS OF THE DRAWING

FIG. 1 is the front view and the length of the textured convex head.

FIG. 1A is the side view and the height of the textured convex head.

FIG. 2 is the length of the handle.

FIG. 2A is the top view and circumference of the handle.

FIG. 3 is the top view of the flat platform with a 0.49 inch hole.

FIG. 3A is the thickness of the wooden platform.

FIG. 4 is the width of the convex head.

FIG. 4A is the total length of the apparatus.

### DETAILED DESCRIPTION OF THE INVENTION

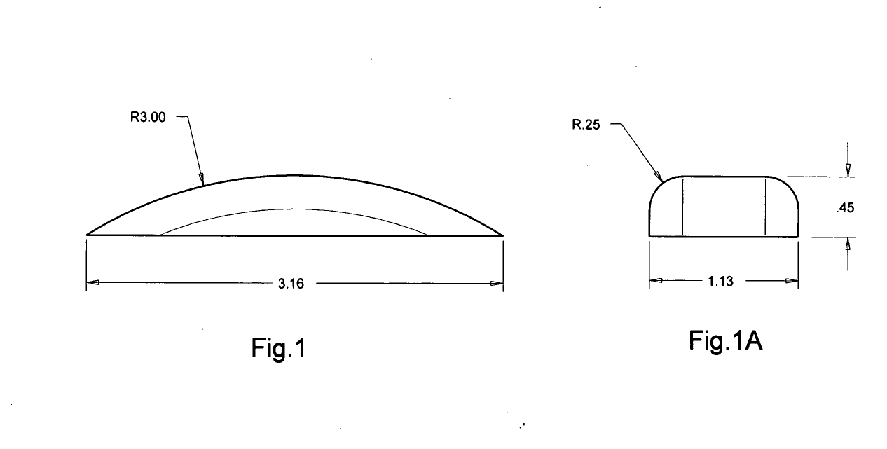

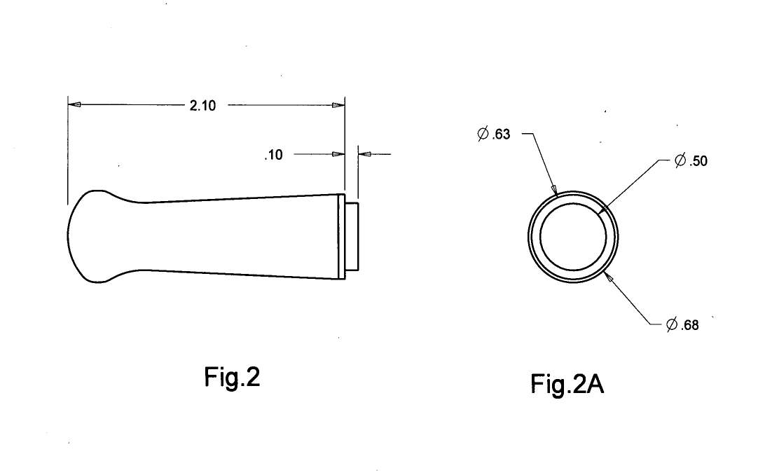

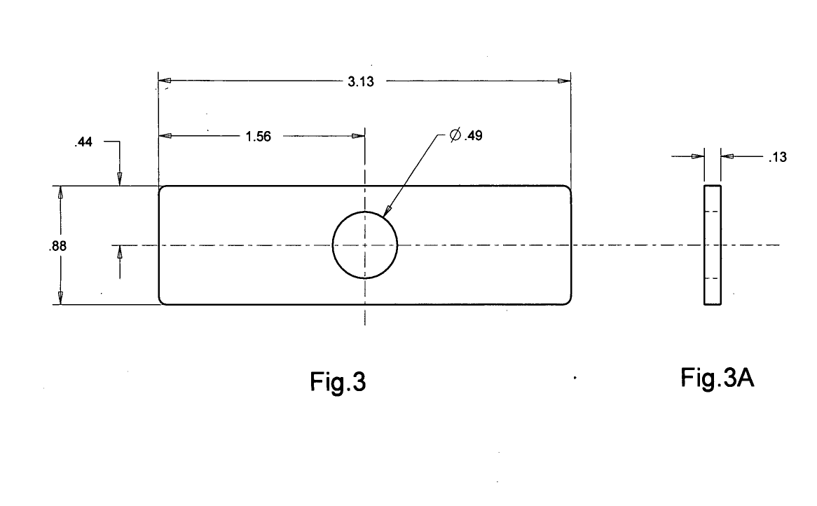

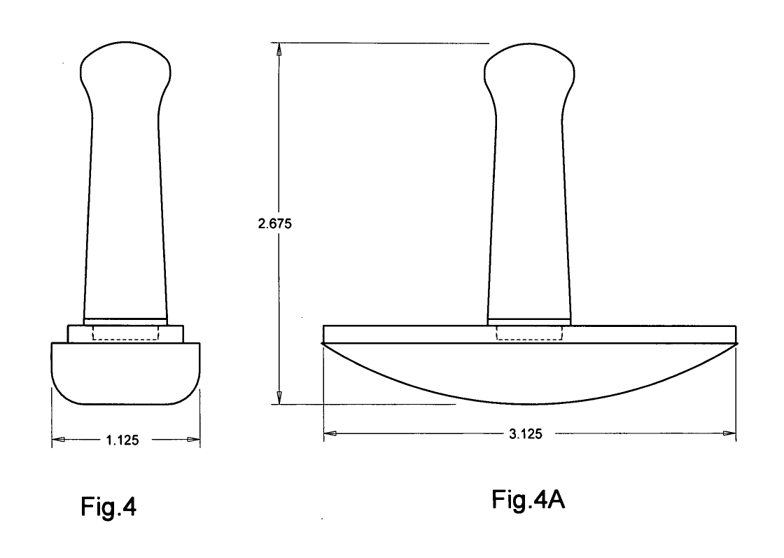

In FIG. 1 a front view of the main part of preferred embodiment is the textured convex head which is 3.16 inches long, with a width of 1.13 inches seen in FIG. 1A, with enough surface area to pull out multiple ingrown hairs that cause razor bumps. The textured convex head has rounded corners and a thickness of 0.45 inches as seen in FIG. 1A for easy movement along the face, neck, arm and pubic area. The handle, which is waterproof, shown in FIG. 2, FIG. 4 and FIG. 4A, is tapered and has a peaked thickness of 0.68 inches shown in 2A. It has a 0.10 inch protruded tip that is inserted inside a 0.49 inch hole in the flat wooden platform, which is shown in FIG. 3. The textured convex head in FIG. 1 is attached to the flat wooden platform with the inserted handle seen in FIG. 4 and FIG. 4A. This enables the device to be moved fluently in multi-directions to dislodge the ingrown hairs from the skin.

The protruding tip at the top of the handle in FIG. 2 is inserted through the flat platform with a thickness of 0.13 inches, shown in FIG. 3A, was determined by the need for structuring strength. The apparatus has to withstand the everyday usage, when pressure is applied in the up and down or side to side motion. In FIG. 4, the side view shows the rounded corners, which allows for a gentle rotation of movement on the skin surface with comfort, by exfoliating and pulling out multiple ingrown hairs simultaneously. The apparatus requires an application of hot water, using soap lather or shaving foam, which opens the pores of the skin and loosens the ingrown hairs that form razor bumps.

What we claimed is:

1. A technique and apparatus for extracting ingrown hairs comprising of:
(a) a textured convex head with rounded corners for fluent movement;
(b) a flat wooden platform with a 0.49 inch hole in the center;
(c) Wooden waterproof handle which supports the flat wooden platform and textured convex head.

2. A technique and apparatus for extracting ingrown hairs in claim 1, wherein the corners of the convex textured head are rounded with a radius of 0.25 inches and the textured head is 1.125 inches wide enough for pulling out multiple ingrown hairs from the skin.

3. A technique and apparatus for extracting ingrown hairs in claim 1b, wherein the 3.25 inch long flat wooden platform has a center hole of 0.49 inches and a thickness of 0.13 inches for structuring support.

4. A technique and apparatus for extracting ingrown hairs in claim 1a and 1b, wherein the textured convex head is attached to the 3.25 inch flat platform with a center hole of 0.49 inches.

5. A technique and apparatus for extracting ingrown hairs in claim 1c, wherein the 2.20 inch handle is attached to the center of the 3.25 inch long flat wooden platform.

6. A technique and apparatus for extracting ingrown hairs in claim 1a, 1b and 1c wherein the 2.20 inch waterproof handle is attached to the 3.25 inch long flat wooden platform and assembled to the textured convex head as a unit.
